(19) United States
(12) Patent Application Publication
(10) Pub. No.: US 2024/0390408 A1
(43) Pub. Date: Nov. 28, 2024

(54) METHOD OF TREATING HEPATITIS C VIRUS IN PATIENTS

(71) Applicant: Altesa BioSciences, Inc., College Park, GA (US)

(72) Inventors: Brett Paul GIROIR, College Station, TX (US); Katherine Elizabeth SQUIRES, Atlanta, GA (US); John Michael CLERICI, Reston, VA (US)

(73) Assignee: Altesa BioSciences, Inc., College Park, GA (US)

(21) Appl. No.: 18/672,122

(22) Filed: May 23, 2024

Related U.S. Application Data
(60) Provisional application No. 63/469,153, filed on May 26, 2023.

(51) Int. Cl. A61K 31/7068; A61K 9/00; A61K 45/06

(52) U.S. Cl. CPC A61K 31/7068; A61K 9/0053; A61K 45/06

(57) ABSTRACT

The present disclosure describes methods of treating a hepatitis C virus (HCV) infection in a patient in need thereof by orally administering to the patient a therapeutically effective amount of a pharmaceutical composition, which may include one or more of compounds disclosed herein. The methods of treating the HCV infection in the patient may result in clearance of the HCV infection as measured by sustained virologic response.

### CROSS REFERENCE TO RELATED APPLICATIONS

This application claims the benefit of priority under 35 U.S.C. § 119 (c) of U.S. Provisional Patent Application Ser. No. 63/469,153 filed on May 26, 2023. The disclosure of the prior application is considered part of and are herein incorporated by reference in the disclosure of this application in their entirety.

### BACKGROUND

The hepatitis C virus (HCV) is a single-stranded, positive sense RNA virus in the family Flaviviridae that infects approximately 170 million people worldwide. There are seven distinct genotypes (GTs) of the hepatitis C virus, which are indicated numerically, with 67 subtypes characterized, with significant variability in their geographic distribution. Genotype 1 (GT1) accounts for 40-80% of all isolates worldwide, though GT2 is also widespread. The hepatitis C virus particle consists of a core of genetic material (RNA), surrounded by an icosahedral protective shell, and further encased in a lipid envelope. Two viral envelope glycoproteins, E1 and E2, are embedded in the lipid envelope. The genome consists of a single open reading frame translated to produce a single protein. This large pre-protein is later cut by cellular and viral proteases into smaller proteins that allow viral replication within the host cell, or assemble into the mature viral particles, e.g., E1, E2, NS2, NS3, NS4, NS4A, NS4B, NS5, NS5A, and NS5B.

HCV leads to inflammation of the liver, and chronic infection can lead to cirrhosis and multiple forms of cancer including liver cancer, lymphoma, and head and neck cancers. Most people with hepatitis C infection have the chronic form. Diagnosis of HCV can occur via nucleic acid analysis of the 5′-noncoding region. ELISA assay may be performed to detect hepatitis C antibodies and RNA assays to determine viral load. Subjects infected with HCV may exhibit symptoms of abdominal pain, ascites, dark urine, fatigue, generalized itching, jaundice, fever, nausea, pale or clay-colored stools and vomiting.

The first generation of interferon-free direct-acting antiviral agents (DAAs) have demonstrated improved sustained virologic response rates in GT1-infected patients, with shorter duration of treatment and improved safety and tolerability related to interferon-containing regimens. However, clinical data suggest that first-generation interferon-free DAAs may not have pan-genotypic activity or efficacy across all subpopulations, may require coadministration other active agents, and may be associated with significant drug-drug interactions. Additionally, subjects who have failed first generation DAA therapy with or without interferon or other active agents are likely to have drug-resistant HCV variants and have limited future treatment options.

Nucleoside and nucleotide phosphates and phosphonates are clinically useful as antiviral agents. However, there remains a need to identify additional effective methods of treating viruses, such as HCV, and dosing regimens for the same.

### SUMMARY

In some aspects, there is provided a method of treating a hepatitis C virus infection in a patient in need thereof including orally administering to the patient in need thereof, a therapeutically effective amount of a compound of Formula I:

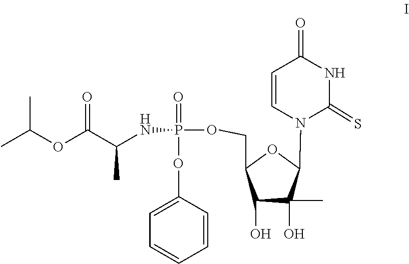

or a pharmaceutically acceptable salt thereof, thereby treating the hepatitis C virus infection in the patient.

In some aspects, the therapeutically effective amount of the compound of Formula I or a pharmaceutically acceptable salt thereof is between about 5 mg/kg and about 400 mg/kg.

In some aspects, the therapeutically effective amount of the compound of Formula I or a pharmaceutically acceptable salt thereof is about 25 mg/kg.

In some aspects, the therapeutically effective amount of the compound of Formula I or a pharmaceutically acceptable salt thereof is about 500 mg to about 2000 mg.

In some aspects, the therapeutically effective amount of the compound of Formula I or a pharmaceutically acceptable salt thereof is administered once a day.

In some aspects, the therapeutically effective amount of the compound of Formula I or a pharmaceutically acceptable salt thereof is administered twice a day.

In some aspects, the therapeutically effective amount of the compound of Formula I or a pharmaceutically acceptable salt thereof is administered over a period of about 2 weeks to about 24 weeks.

In some aspects, the hepatitis C virus infection is selected from the group consisting of Genotype 1, Genotype 2, Genotype 3, Genotype 4, Genotype 5, or Genotype 6, or any combination thereof.

In some aspects, the hepatitis C virus infection is a strain selected from the group consisting of 1a, 1b, 1b/2b NS5B, 1b/4a NS5B, 1b/NS5B S282T, 1b/NS5B S96T, 1b/3a NS5B, or any combination thereof.

In some aspects, the patient has compensated cirrhosis.

In some aspects, the patient has decompensated cirrhosis.

In some aspects, the patient does not have cirrhosis.

In some aspects, administering the therapeutically effective amount of the compound of Formula I or a pharmaceutically acceptable salt thereof results in clearance of the hepatitis C virus infection in the patient.

In some aspects, administering the therapeutically effective amount of the compound of Formula I or a pharmaceutically acceptable salt thereof results in a sustained virologic response after about 12 weeks to about 24 weeks of treatment.

In some aspects, the method further includes administering a second active agent for the treatment of the hepatitis C virus infection.

In some aspects, the second active agent for the treatment of the hepatitis C virus infection includes sofosbuvir, ledipasvir, velpatasvir, elbasvir, grazoprevir, glecaprevir, pibrentasvir, voxelaprevir, ribavirin, boceprevir, daclatasvir, ombitasvir, paritaprevir, ritonavir, dasabuvir, simeprevir, telaprevir, or combinations thereof.

In some aspects, the method further includes administering a third active agent for the treatment of the hepatitis C virus infection.

In some aspects, the third active agent for the treatment of the hepatitis C virus infection includes sofosbuvir, ledipasvir, velpatasvir, elbasvir, grazoprevir, glecaprevir, pibrentasvir, voxelaprevir, ribavirin, boceprevir, daclatasvir, ombitasvir, paritaprevir, ritonavir, dasabuvir, simeprevir, telaprevir, or combinations thereof.

In some aspects, there is provided a method of treating a hepatitis C virus infection in a patient in need thereof including orally administering to the patient a therapeutically effective amount of a compound of Formula I:

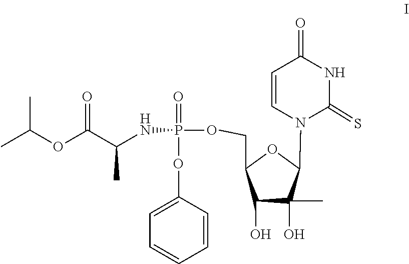

or a pharmaceutically acceptable salt thereof, wherein administering a therapeutically effective amount of the compound of Formula I or a pharmaceutically acceptable salt thereof results in clearance of the hepatitis C virus infection.

In some aspects, clearance of the hepatitis C virus infection includes a sustained virologic response after about 12 weeks to about 24 weeks of treatment.

In some aspects, the therapeutically effective amount of the compound of Formula I or a pharmaceutically acceptable salt thereof is between about 5 mg/kg and about 400 mg/kg.

In some aspects, the therapeutically effective amount of the compound of Formula I or a pharmaceutically acceptable salt thereof is about 25 mg/kg.

In some aspects, the therapeutically effective amount of the compound of Formula I or a pharmaceutically acceptable salt thereof is about 500 mg to about 2000 mg.

In some aspects, the therapeutically effective amount of the compound of Formula I or a pharmaceutically acceptable salt thereof is administered once a day.

In some aspects, the therapeutically effective amount of the compound of Formula I or a pharmaceutically acceptable salt thereof is administered twice a day.

In some aspects, the hepatitis C virus infection is selected from the group consisting of Genotype 1, Genotype 2, Genotype 3, Genotype 4, Genotype 5, or Genotype 6, or any combination thereof.

In some aspects, the hepatitis C virus infection is a strain selected from the group consisting of 1a, 1b, 1b/2b NS5B, 1b/4a NS5B, 1b/NS5B S282T, 1b/NS5B S96T, 1b/3a NS5B, or any combination thereof.

In some aspects, the patient has compensated cirrhosis.

In some aspects, the patient has decompensated cirrhosis.

In some aspects, the patient does not have cirrhosis.

In some aspects, the method further includes administering a second active agent for the treatment of the hepatitis C virus infection.

In some aspects, the second active agent for the treatment of the hepatitis C virus infection includes sofosbuvir, ledipasvir, velpatasvir, elbasvir, grazoprevir, glecaprevir, pibrentasvir, voxelaprevir, ribavirin, boceprevir, daclatasvir, ombitasvir, paritaprevir, ritonavir, dasabuvir, simeprevir, telaprevir, or combinations thereof.

In some aspects, the method further includes administering a third active agent for the treatment of the hepatitis C virus infection.

In some aspects, the third active agent for the treatment of the hepatitis C virus infection includes sofosbuvir, ledipasvir, velpatasvir, elbasvir, grazoprevir, glecaprevir, pibrentasvir, voxelaprevir, ribavirin, boceprevir, daclatasvir, ombitasvir, paritaprevir, ritonavir, dasabuvir, simeprevir, telaprevir, or combinations thereof.

In some aspects, there is provided a method of treating a hepatitis C virus infection in a patient in need thereof comprising orally administering to the patient a prodrug of Formula I:

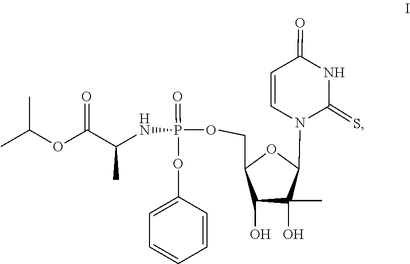

or a pharmaceutically acceptable salt thereof; wherein the prodrug is administered in an amount sufficient to result in a therapeutically effective amount of one or more of a compound of Formula II-VI in the liver of the patient:

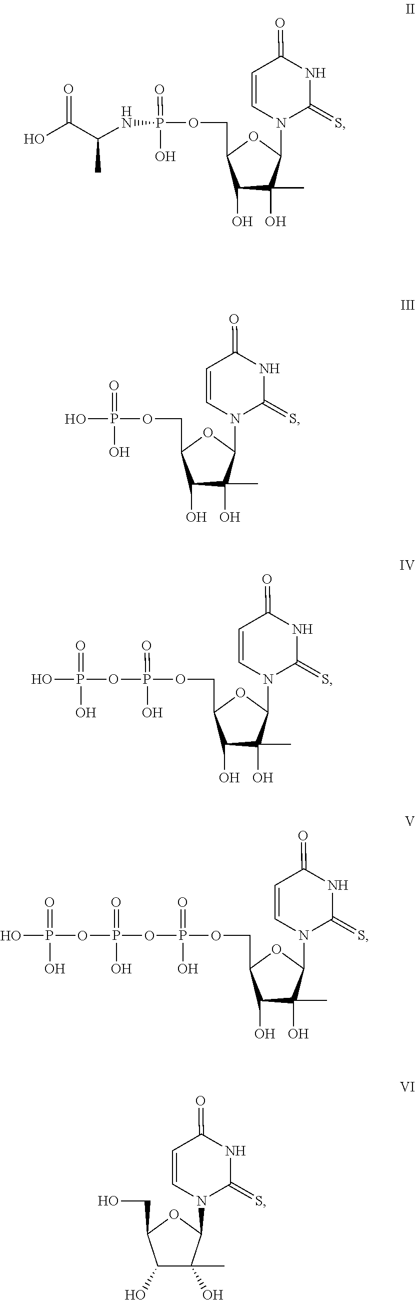

thereby treating the hepatitis C virus infection in the patient.

In some aspects, the amount of the prodrug of Formula I or a pharmaceutically acceptable salt thereof is between about 5 mg/kg and about 400 mg/kg.

In some aspects, the amount of the prodrug of Formula I or a pharmaceutically acceptable salt thereof is about 25 mg/kg.

In some aspects, the therapeutically effective amount of the prodrug of Formula I or a pharmaceutically acceptable salt thereof is about 500 mg to about 2000 mg.

In some aspects, the prodrug of Formula I or a pharmaceutically acceptable salt thereof is administered once a day.

In some aspects, the prodrug of Formula I or a pharmaceutically acceptable salt thereof is administered twice a day.

In some aspects, the prodrug of Formula I or a pharmaceutically acceptable salt thereof is administered over a period of about 2 weeks to about 24 weeks.

In some aspects, the hepatitis C virus infection is selected from the group consisting of Genotype 1, Genotype 2, Genotype 3, Genotype 4, Genotype 5, Genotype 6, or any combination thereof.

In some aspects, the hepatitis C virus infection is a strain selected from the group consisting of 1a, 1b, 1b/2b NS5B, 1b/4a NS5B, 1b/NS5B S282T, 1b/NS5B S96T, 1b/3a NS5B, or any combination thereof.

In some aspects, the patient has compensated cirrhosis.

In some aspects, the patient has decompensated cirrhosis.

In some aspects, the patient does not have cirrhosis.

In some aspects, administering the prodrug of Formula I or a pharmaceutically acceptable salt thereof results in clearance of the hepatitis C virus infection.

In some aspects, administering the prodrug of Formula I or a pharmaceutically acceptable salt thereof results in a sustained virologic response after about 12 weeks to about 24 weeks of treatment.

In some aspects, the method further includes administering a second active agent for the treatment of the hepatitis C virus infection.

In some aspects, the second active agent for the treatment of the hepatitis C virus infection includes sofosbuvir, ledipasvir, velpatasvir, elbasvir, grazoprevir, glecaprevir, pibrentasvir, voxelaprevir, ribavirin, boceprevir, daclatasvir, ombitasvir, paritaprevir, ritonavir, dasabuvir, simeprevir, telaprevir, or combinations thereof.

In some aspects, the method further includes administering a third active agent for the treatment of the hepatitis C virus infection.

In some aspects, the third active agent for the treatment of the hepatitis C virus infection includes sofosbuvir, ledipasvir, velpatasvir, elbasvir, grazoprevir, glecaprevir, pibrentasvir, voxelaprevir, ribavirin, boceprevir, daclatasvir, ombitasvir, paritaprevir, ritonavir, dasabuvir, simeprevir, telaprevir, or combinations thereof.

### DETAILED DESCRIPTION

This disclosure relates generally to methods of treating viruses, such as hepatitis C virus (HCV) with nucleotide and nucleoside therapeutic compositions. In certain embodiments, the disclosure relates to methods of treating a viral infection including administering a therapeutically effective amount of a compound disclosed herein to a subject in need thereof.

Before compounds, compositions and methods are described in detail, it is to be understood that this disclosure is not limited to the particular processes, compositions, or methodologies described, as these may vary. It is also to be understood that the terminology used in the description is for the purpose of describing the particular versions or embodiments only, and is not intended to limit the scope of the disclosure which will be limited only by the appended claims. Unless defined otherwise, all technical and scientific terms used herein have the same meaning as commonly understood by one of ordinary skill in the art. Although any methods and materials similar or equivalent to those described herein can be used in the practice or testing of embodiments of the disclosure, the preferred methods, devices, and materials are now described.

It is further appreciated that certain features of the disclosure, which are, for clarity, described in the context of separate embodiments, can also be provided in combination in a single embodiment. Conversely, various features of the disclosure which are, for brevity, described in the context of a single embodiment, can also be provided separately or in any suitable subcombination.

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention. As used in this document, the term “comprising” means “including, but not limited to.”

As used herein, the term “about” means plus or minus up to 20% of the numerical value of the number with which it is being used. For example, “about 50%” means in the range of 40-60% and includes exactly 50%. The term “about” may refer to plus or minus 1%, 5%, 10%, 15%, or 20% of the numerical value of the number with which it is being used.

“Administering,” when used in conjunction with the compounds of the disclosure, means to administer a compound directly into or onto a target tissue or to administer a compound systemically or locally to a patient or other subject.

The term “animal” as used herein includes, but is not limited to, humans and non-human vertebrates such as wild, experimental, domestic, and farm animals, and pets. As used herein, the terms “subject,” “individual,” and “patient,” are used interchangeably and refer to any animal, including mammals, mice, rats, other rodents, rabbits, dogs, cats, swine, cattle, sheep, horses, primates, non-human primates, humans, and the like.

The term “improves” is used to convey that the disclosure changes either the characteristics and/or the physical attributes of the tissue to which it is being provided, applied or administered. The term “improves” may also be used in conjunction with a disease state such that when a disease state is “improved” the symptoms or physical characteristics associated with the disease state are diminished, reduced, eliminated, delayed, or averted.

The term “inhibiting” includes the blockade, aversion of a certain result or process, or the restoration of the converse result or process. In terms of treatment by administration of a compound of the disclosure, “inhibiting” includes protecting against (partially or wholly) or delaying the onset of symptoms, alleviating symptoms, or protecting against, diminishing or eliminating a disease, condition, or disorder.

As used herein, the term “free base” refers to a non-salt form of a compound as described herein.

The term “salts” can include acid addition salts or addition salts of free bases. The salts as described herein may be pharmaceutically acceptable. Examples of acids which may be employed to form pharmaceutically acceptable acid addition salts include but are not limited to salts derived from nontoxic inorganic acids such as nitric, phosphoric, sulfuric, hydrobromic, hydroiodic, hydrofluoric, or phosphorus acids, as well as salts derived from nontoxic organic acids such as aliphatic mono- and dicarboxylic acids, phenyl-substituted alkanoic acids, hydroxyl alkanoic acids, alkanedioic acids, aromatic acids, aliphatic and aromatic sulfonic acids, and acetic, maleic, succinic, or citric acids. Non-limiting examples of such salts include napadisylate, besylate, sulfate, pyrosulfate, bisulfate, sulfite, bisulfite, nitrate, phosphate, monohydrogenphosphate, dihydrogenphosphate, metaphosphate, pyrophosphate, chloride, hydrochloride, bromide, iodide, acetate, trifluoroacetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, maleate, tartrate, methanesulfonate, and the like. Salts of amino acids are also contemplated, such as arginate, gluconate, galacturonate, and the like.

Those skilled in the art of organic chemistry will appreciate that many organic compounds can form complexes with solvents in which they are reacted or from which they are precipitated or crystallized. For example, a complex with water is known as a “hydrate.” Solvates of the compounds of the present disclosure are within the scope of this disclosure. The salts of the compound of any of the formulae described herein may form solvates (e.g., hydrates) and the present disclosure includes all such solvates. The meaning of the word “solvates” is well known to those skilled in the art as a compound formed by interaction of a solvent and a solute (i.e., solvation). Techniques for the preparation of solvates are well established in the art.

As used herein, unless specifically indicated, the term “active ingredient” refers to a compound of any of the formulae as described herein.

The phrase “pharmaceutically acceptable” refers to molecular entities and compositions that are generally regarded as safe and nontoxic. In particular, pharmaceutically acceptable carriers, diluents or other excipients used in the pharmaceutical compositions of this disclosure are physiologically tolerable, compatible with other ingredients, and do not typically produce an allergic or similar untoward reaction (for example, gastric upset, dizziness and the like) when administered to a subject. In some embodiments, as used herein, the term “pharmaceutically acceptable” means approved by a regulatory agency of the Federal or a state government or listed in the U.S. Pharmacopoeia or other generally recognized pharmacopocia for use in animals, and more particularly in humans. The phrase “pharmaceutically acceptable salt(s)”, as used herein, includes those salts of compounds of the disclosure that are safe and effective for use in mammals and that possess the desired biological activity. Pharmaceutically acceptable salts include salts of acidic or basic groups present in compounds of the disclosure or in compounds identified pursuant to the methods of the disclosure. Pharmaceutically acceptable acid addition salts include, but are not limited to, hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate, and pamoate (i.e., 1, l′-methylene-bis-(2-hydroxy-3-naphthoate)) salts. Certain compounds of the disclosure can form pharmaceutically acceptable salts with various amino acids. Suitable base salts include, but are not limited to, aluminum, calcium, lithium, magnesium, potassium, sodium, zinc, iron and diethanolamine salts. Pharmaceutically acceptable base addition salts are also formed with amines, such as organic amines. Examples of suitable amines are N,N′-dibenzylethylenediamine, chloroprocaine, choline, diethanolamine, dicyclohexylamine, ethylenediamine, N-methylglucamine, and procaine.

As used herein, the term “therapeutic” means an agent utilized to treat, combat, ameliorate, protect against, or improve an unwanted condition or disease of a subject.

A “therapeutically effective amount” or “effective amount” of a compound or composition of the disclosure is a predetermined amount which confers a therapeutic effect on the treated subject, at a reasonable benefit/risk ratio applicable to any medical treatment. The therapeutic effect may be objective (i.e., measurable by some test or marker) or subjective (i.e., subject gives an indication of or feels an effect or physician observes a change). The effect contemplated herein includes medical therapeutic treatment, as appropriate. The specific dose of a compound administered according to this disclosure to obtain therapeutic effects will, of course, be determined by the particular circumstances surrounding the case, including, for example, the compound administered, the route of administration, the co-administration of other active ingredients, the condition being treated, the activity of the specific compound employed, the specific composition employed, the age, body weight, general health, sex and diet of the subject; the time of administration, route of administration, and rate of excretion of the specific compound employed and the duration of the treatment. The effective amount administered will be determined by the physician in the light of the foregoing relevant circumstances and the exercise of sound medical judgment. A therapeutically effective amount of a compound of this disclosure is typically an amount such that when it is administered in a physiologically tolerable excipient composition, it is sufficient to achieve an effective systemic concentration or local concentration in the tissue.

The terms “treat”, “treated”, or “treating” as used herein refers to therapeutic treatment measures, wherein the object is to protect against (partially or wholly) or slow down (e.g., lessen or postpone the onset of) an undesired physiological condition, disorder or disease, or to obtain beneficial or desired clinical results such as partial or total restoration or inhibition in decline of a parameter, value, function or result that had or would become abnormal. For the purposes of this disclosure, beneficial or desired clinical results include, but are not limited to, alleviation of symptoms; diminishment of the extent or vigor or rate of development of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether or not it translates to immediate lessening of actual clinical symptoms, or enhancement or improvement of the condition, disorder or disease. Treatment seeks to elicit a clinically significant response without excessive levels of side effects. Treatment also includes prolonging survival as compared to expected survival if not receiving treatment.

As used herein, the term “prodrug” refers to a pharmacologically inactive compound which, when administered to a patient, is metabolized into a pharmacologically active compound.

As used herein, the abbreviation “BID” means bis in de and refers to twice a day dosage.

Unless otherwise indicated, the abbreviations “h” and “hr” refer to hours as a unit of time.

The present disclosure describes methods of treating a hepatitis C virus infection in a patient in need thereof using a compound of Formula I, as shown below. The compound of Formula I has a molecular weight of 543.54 and molecular formula C₂₂H₃₀N₃O₉PS.

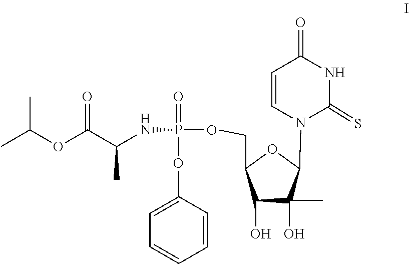

In any embodiment described herein, the compound of Formula I may be limited to a single diastereomer, such as the compound of Formula I (a):

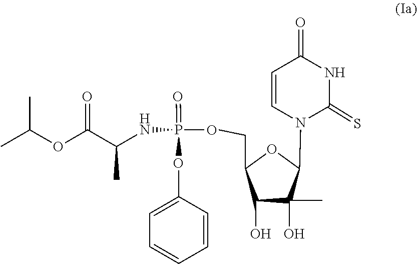

Or the compound of Formula I (b):

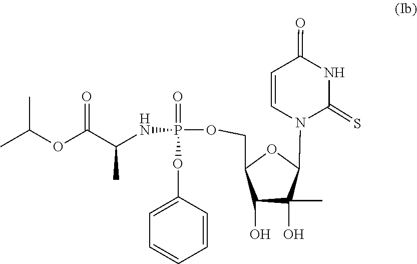

In any embodiment described herein, the compound of Formula I may be present as a mixture of the compounds of Formula I (a) and Formula I (b). The ratio of such a mixture is not particularly limited and may be any ratio, such as 99:1, 90:10, 80:20, 70:30, 60:40, 50:50, 40:60, 30:70, 20:80, 10:90, 1:99, or any range or value contained therein.

There is provided a method of treating a hepatitis C virus infection in a patient in need thereof, wherein the method includes orally administering to a patient in thereof a therapeutically effective amount of the compound of Formula I or a pharmaceutically acceptable salt thereof, thereby treating the hepatitis C virus infection in the patient.

In any embodiment described herein, the therapeutically effective amount of the compound of Formula I or a pharmaceutically acceptable salt thereof is between about 5 mg/kg and about 400 mg/kg. For example, the therapeutically effective amount of the compound of Formula I or a pharmaceutically acceptable salt thereof may be about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, about 105 mg/kg, about 110 mg/kg, about 115 mg/kg, about 120 mg/kg, about 125 mg/kg, about 130 mg/kg, about 135 mg/kg, about 140 mg/kg, about 145 mg/kg, about 150 mg/kg, about 155 mg/kg, about 160 mg/kg, about 165 mg/kg, about 170 mg/kg, about 175 mg/kg, about 180 mg/kg, about 185 mg/kg, about 190 mg/kg, about 195 mg/kg, about 200 mg/kg, about 250 mg/kg, about 300 mg/kg, about 350 mg/kg, about 400 mg/kg, or any range or value contained therein.

In some embodiments, the therapeutically effective amount of the compound of Formula I or a pharmaceutically acceptable salt thereof is about 500 mg to about 2000 mg, or any range or value contained therein. Doses of about 5 mg, about 10 mg, about 50 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, about 1200 mg, about 1250 mg, about 1300 mg, about 1350 mg, about 1400 mg, about 1450 mg, about 1500 mg, about 1550 mg, about 1600 mg, about 1650 mg, about 1700 mg, about 1750 mg, about 1800 mg, about 1850 mg, about 1900 mg, about 1950 mg, about 2000 mg, and any range or value contained therein are also contemplated.

In any embodiment described herein, the therapeutically effective amount of the compound of Formula I or a pharmaceutically acceptable salt thereof is administered once a day (QD). In some embodiments, the therapeutically effective amount of the compound of Formula I or a pharmaceutically acceptable salt thereof is administered twice a day (BID).

In any embodiment described herein, the therapeutically effective amount of the compound of Formula I or a pharmaceutically acceptable salt thereof is administered over a period of about 2 weeks to about 24 weeks, such as about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, about 21 weeks, about 22 weeks, about 23 weeks, about 24 weeks, or any range or value contained therein. Throughout the period during which the therapeutically effective amount of the compound of Formula I or a pharmaceutically acceptable salt thereof is administered, the therapeutically effective amount may be administered once a day, twice a day, or combinations thereof over the period of about 2 weeks to about 24 weeks.

In any embodiment described herein, the hepatitis C virus infection is selected from the group consisting of Genotype 1, Genotype 2, Genotype 3, Genotype 4, Genotype 5, Genotype 6, or any combination thereof. In some embodiments, the hepatitis C virus infection is a strain selected from the group consisting of 1a, 1b, 1b/2b NS5B, 1b/4a NS5B, 1b/NS5B S282T, 1b/NS5B S96T, 1b/3a NS5B, or any combination thereof.

In any embodiment described herein, the patient is a human. The age and gender of the patient are not particularly limited.

In any embodiment described herein, the patient has cirrhosis, which may be compensated or decompensated. The cirrhosis may be asymptomatic or substantially asymptomatic. In some embodiments, the patient does not have cirrhosis.

In any embodiment described herein, administering the therapeutically effective amount of the compound of Formula I or a pharmaceutically acceptable salt thereof results in a sustained virologic response after about 12 weeks to about 24 weeks of treatment.

In any embodiment described herein, the method of treating a hepatitis C virus infection in a patient in need thereof further includes administering a second active agent for the treatment of the hepatitis C virus infection. The method may, in some embodiments, include administering a second active agent for the treatment of the hepatitis C virus infection and a third active agent for the treatment of the hepatitis C virus infection. It is contemplated that the method of treating a hepatitis C virus infection may include administering the compound of Formula I for the treatment of the hepatitis C virus infection along with a second active agent, a third active agent, a fourth active agent, and so on. Examples of suitable second active agents and third active agents for the treating of a hepatitis C virus infection in a patient include, but are not limited to, NS5A inhibitors, protease inhibitors such as NS3/4 protease inhibitors, NS5B polymerase inhibitors, and interferons. Examples of suitable second activate agents and third active agents include, but are not limited to, sofosbuvir, ledipasvir, velpatasvir, elbasvir, grazoprevir, glecaprevir, pibrentasvir, voxelaprevir, ribavirin, boceprevir, daclatasvir, ombitasvir, paritaprevir, ritonavir, dasabuvir, simeprevir, telaprevir, and combinations thereof. Other active agents for the treatment of a hepatitis C virus infection known to those skilled in the art are also within the scope of this disclosure. In any aspect of the present disclosure, the second active agent for the treatment of the hepatitis C virus infection includes sofosbuvir, ledipasvir, velpatasvir, elbasvir, grazoprevir, glecaprevir, pibrentasvir, voxelaprevir, ribavirin, boceprevir, daclatasvir, ombitasvir, paritaprevir, ritonavir, dasabuvir, simeprevir, telaprevir, or combinations thereof. In any aspect of the present disclosure, the third active agent for the treatment of the hepatitis C virus infection includes sofosbuvir, ledipasvir, velpatasvir, elbasvir, grazoprevir, glecaprevir, pibrentasvir, voxelaprevir, ribavirin, boceprevir, daclatasvir, ombitasvir, paritaprevir, ritonavir, dasabuvir, simeprevir, telaprevir, or combinations thereof.

There is provided a method of treating a hepatitis C virus infection in a patient in need thereof which includes orally administering to a patient a therapeutically effective amount of a compound of Formula I:

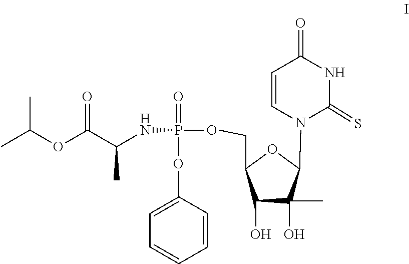

or a pharmaceutically acceptable salt thereof, wherein administering the therapeutically effective amount of the compound of Formula I or a pharmaceutically acceptable salt thereof results in clearance of the hepatitis C virus infection.

In any embodiment described herein, clearance of the hepatitis C virus infection includes a sustained virologic response after about 12 weeks to about 24 weeks of treatment. Clearance of the hepatitis C virus infection may further include a reduction or elimination of symptoms of the hepatitis C virus infection.

In any embodiment described herein, the therapeutically effective amount of the compound of Formula I or a pharmaceutically acceptable salt thereof of the methods described herein that results in clearance of the hepatitis C virus infection is between about 5 mg/kg and about 400 mg/kg. For example, the therapeutically effective amount of the compound of Formula I or a pharmaceutically acceptable salt thereof may be about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, about 105 mg/kg, about 110 mg/kg, about 115 mg/kg, about 120 mg/kg, about 125 mg/kg, about 130 mg/kg, about 135 mg/kg, about 140 mg/kg, about 145 mg/kg, about 150 mg/kg, about 155 mg/kg, about 160 mg/kg, about 165 mg/kg, about 170 mg/kg, about 175 mg/kg, about 180 mg/kg, about 185 mg/kg, about 190 mg/kg, about 195 mg/kg, about 200 mg/kg, about 250 mg/kg, about 300 mg/kg, about 350 mg/kg, about 400 mg/kg, or any range or value contained therein.

In some embodiments, the therapeutically effective amount of the compound of Formula I or a pharmaceutically acceptable salt thereof is about 500 mg to about 2000 mg, or any range or value contained therein. Doses of about 5 mg, about 10 mg, about 50 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, about 1200 mg, about 1250 mg, about 1300 mg, about 1350 mg, about 1400 mg, about 1450 mg, about 1500 mg, about 1550 mg, about 1600 mg, about 1650 mg, about 1700 mg, about 1750 mg, about 1800 mg, about 1850 mg, about 1900 mg, about 1950 mg, about 2000 mg, and any range or value contained therein are also contemplated.

In any embodiment described herein, the method of treating the hepatitis C virus infection in the patient that results in clearance of the hepatitis C virus infection in the patient includes administering the therapeutically effective amount of the compound of Formula I or a pharmaceutically acceptable salt thereof once a day (QD). In some embodiments, the therapeutically effective amount of the compound of Formula I or a pharmaceutically acceptable salt thereof is administered twice a day (BID).

In any embodiment described herein, the therapeutically effective amount of the compound of Formula I or a pharmaceutically acceptable salt thereof is administered over a period of about 2 weeks to about 24 weeks, such as about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, about 21 weeks, about 22 weeks, about 23 weeks, about 24 weeks, or any range or value contained therein. Throughout the period during which the therapeutically effective amount of the compound of Formula I or a pharmaceutically acceptable salt thereof is administered, the therapeutically effective amount may be administered once a day, twice a day, or combinations thereof over the period of about 2 weeks to about 24 weeks.

In any embodiment described herein, the hepatitis C virus infection is selected from the group consisting of Genotype 1, Genotype 2, Genotype 3, Genotype 4, Genotype 5, Genotype 6, or any combination thereof. In some embodiments, the hepatitis C virus infection is a strain selected from the group consisting of 1a, 1b, 1b/2b NS5B, 1b/4a NS5B, 1b/NS5B S282T, 1b/NS5B S96T, 1b/3a NS5B, or any combination thereof.

In any embodiment described herein, the patient is a human. The age and gender of the patient are not particularly limited.

In any embodiment described herein, the patient has cirrhosis, which may be compensated or decompensated. The cirrhosis may be asymptomatic or substantially asymptomatic. In some embodiments, the patient does not have cirrhosis.

In any embodiment described herein, administering the therapeutically effective amount of the compound of Formula I or a pharmaceutically acceptable salt thereof results in a sustained virologic response after about 12 weeks to about 24 weeks of treatment.

In any embodiment described herein, the method of treating a hepatitis C virus infection in a patient in need thereof further includes administering a second active agent for the treatment of the hepatitis C virus infection. The method may, in some embodiments, include administering a second active agent for the treatment of the hepatitis C virus infection and a third active agent for the treatment of the hepatitis C virus infection. It is contemplated that the method of treating a hepatitis C virus infection may include administering the compound of Formula I or a pharmaceutically acceptable salt thereof for the treatment of the hepatitis C virus infection along with a second active agent, a third active agent, a fourth active agent, and so on. Examples of suitable second active agents and third active agents for the treating of a hepatitis C virus infection in a patient include, but are not limited to, NS5A inhibitors, protease inhibitors such as NS3/4 protease inhibitors, NS5B polymerase inhibitors, and interferons. Examples of suitable second activate agents and third active agents include, but are not limited to, sofosbuvir, ledipasvir, velpatasvir, elbasvir, grazoprevir, glecaprevir, pibrentasvir, voxelaprevir, ribavirin, boceprevir, daclatasvir, ombitasvir, paritaprevir, ritonavir, dasabuvir, simeprevir, telaprevir, and combinations thereof. Other active agents for the treatment of a hepatitis C virus infection known to those skilled in the art are also within the scope of this disclosure.

There is provided a method of treating a hepatitis C virus infection in a patient in need thereof which includes orally administering to the patient a prodrug of Formula I:

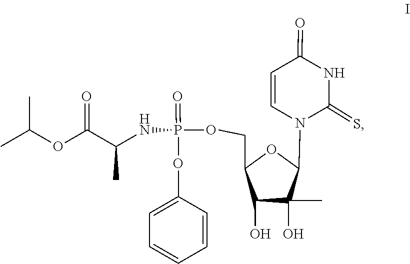

or a pharmaceutically acceptable salt thereof: wherein the prodrug is administered in an amount sufficient to result in a therapeutically effective amount of one or more of a compound of Formula II-VI in the liver of the patient:

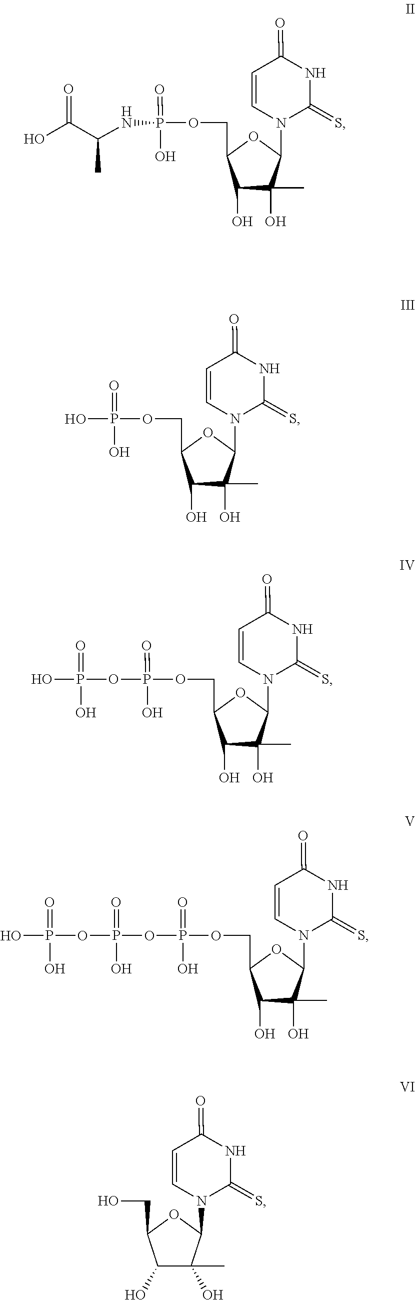

thereby treating the hepatitis C virus infection in the patient.

In some embodiments, the amount of the prodrug of Formula I or a pharmaceutically acceptable salt thereof is between about 5 mg/kg and about 400 mg/kg. In some embodiments, the amount of the prodrug of Formula I or a pharmaceutically acceptable salt thereof is about 125 mg/kg. In some embodiments, the prodrug of Formula I or a pharmaceutically acceptable salt thereof is administered once a day. In some embodiments, the amount of the prodrug of Formula I or a pharmaceutically acceptable salt thereof is administered twice a day. In some embodiments, the amount of the prodrug of Formula I or a pharmaceutically acceptable salt thereof is administered over a period of about 2 weeks to about 24 weeks.

In some embodiments, the hepatitis C virus infection is selected from the group consisting of Genotype 1, Genotype 2, Genotype 3, Genotype 4, Genotype 5, Genotype 6, or any combination thereof. In some embodiments, the hepatitis C virus infection is a strain selected from the group consisting of 1a, 1b, 1b/2b NS5B, 1b/4a NS5B, 1b/NS5B S282T, 1b/NS5B S96T, 1b/3a NS5B, or any combination thereof. In some embodiments, the patient has compensated cirrhosis. In some embodiments, the patient has decompensated cirrhosis. In some embodiments, the patient does not have cirrhosis.

In some embodiments, administering the prodrug of Formula I or a pharmaceutically acceptable salt thereof results in clearance of the hepatitis C virus infection. In some embodiments, administering the prodrug of Formula I or a pharmaceutically acceptable salt thereof results in a sustained virologic response after about 12 weeks to about 24 weeks of treatment. In some embodiments, the method further includes administering a second active agent for the treatment of the hepatitis C virus infection.

In any embodiment described herein, the methods described herein may include administering a prodrug of Formula I or a pharmaceutically acceptable salt thereof which, upon administration to the patient, is metabolized to any of the compounds of Formula II, Formula III, Formula IV, Formula V, and Formula VI. It is contemplated that there may also be other prodrugs which when administered to the patient may result in systemic exposure of one or more of the compounds of Formula I, Formula II, Formula III, Formula IV, Formula V, and Formula VI to the patient; such structures or prodrugs are within the scope of this disclosure.

In any embodiment described herein, the prodrug of Formula I or a pharmaceutically acceptable salt thereof as described herein is administered over a period of about 2 weeks to about 24 weeks, such as about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, about 21 weeks, about 22 weeks, about 23 weeks, about 24 weeks, or any range or value contained therein.

In any embodiment described herein, the prodrug of Formula I or a pharmaceutically acceptable salt thereof as described herein is administered once a day (QD). In some embodiments, the therapeutically effective amount of the pharmaceutical composition as described herein is administered twice a day (BID).

In any embodiment described herein, the therapeutically effective amount of the prodrug of Formula I or a pharmaceutically acceptable salt thereof as described herein is between about 5 mg/kg and about 400 mg/kg. For example, the therapeutically effective amount of the compound of Formula I or a pharmaceutically acceptable salt thereof may be about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, about 105 mg/kg, about 110 mg/kg, about 115 mg/kg, about 120 mg/kg, about 125 mg/kg, about 130 mg/kg, about 135 mg/kg, about 140 mg/kg, about 145 mg/kg, about 150 mg/kg, about 155 mg/kg, about 160 mg/kg, about 165 mg/kg, about 170 mg/kg, about 175 mg/kg, about 180 mg/kg, about 185 mg/kg, about 190 mg/kg, about 195 mg/kg, about 200 mg/kg, about 250 mg/kg, about 300 mg/kg, about 350 mg/kg, about 400 mg/kg, or any range or value contained therein.

In any embodiment described herein, the therapeutically effective amount of the prodrug of Formula I or a pharmaceutically acceptable salt thereof is about 500 mg to about 2000 mg, or any range or value contained therein. Doses of about 5 mg, about 10 mg, about 50 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, about 1200 mg, about 1250 mg, about 1300 mg, about 1350 mg, about 1400 mg, about 1450 mg, about 1500 mg, about 1550 mg, about 1600 mg, about 1650 mg, about 1700 mg, about 1750 mg, about 1800 mg, about 1850 mg, about 1900 mg, about 1950 mg, about 2000 mg, and any range or value contained therein are also contemplated.

In any embodiment described herein, the hepatitis C virus infection is selected from the group consisting of Genotype 1, Genotype 2, Genotype 3, Genotype 4, Genotype 5, Genotype 6, or any combination thereof. In some embodiments, the hepatitis C virus infection is a strain selected from the group consisting of 1a, 1b, 1b/2b NS5B, 1b/4a NS5B, 1b/NS5B S282T, 1b/NS5B S96T, 1b/3a NS5B, or any combination thereof.

In any embodiment described herein, the patient is a human. The age and gender of the patient are not particularly limited.

In any embodiment described herein, the patient has cirrhosis, which may be compensated or decompensated. The cirrhosis may be asymptomatic or substantially asymptomatic. In some embodiments, the patient does not have cirrhosis.

In any embodiment described herein, administering the therapeutically effective amount of the pharmaceutical composition as described herein results in a sustained virologic response after about 12 weeks to about 24 weeks of treatment.

In any embodiment described herein, the method of treating a hepatitis C virus infection in a patient in need thereof further includes administering a second active agent for the treatment of the hepatitis C virus infection. The method may, in some embodiments, include administering a second active agent for the treatment of the hepatitis C virus infection and a third active agent for the treatment of the hepatitis C virus infection. It is contemplated that the method of treating a hepatitis C virus infection may include administering the pharmaceutical composition as described herein for the treatment of the hepatitis C virus infection along with a second active agent, a third active agent, a fourth active agent, and so on. Examples of suitable second active agents and third active agents for the treating of a hepatitis C virus infection in a patient include, but are not limited to, NS5A inhibitors, protease inhibitors such as NS3/4 protease inhibitors, NS5B polymerase inhibitors, and interferons. Examples of suitable second activate agents and third active agents include, but are not limited to, sofosbuvir, ledipasvir, velpatasvir, elbasvir, grazoprevir, glecaprevir, pibrentasvir, voxelaprevir, ribavirin, boceprevir, daclatasvir, ombitasvir, paritaprevir, ritonavir, dasabuvir, simeprevir, telaprevir, and combinations thereof. Other active agents for the treatment of a hepatitis C virus infection known to those skilled in the art, including those in development belonging to multiple different pharmaceutical classes, are also within the scope of this disclosure.

There is provided a method of treating a hepatitis C virus infection in a patient in need thereof which includes performing a diagnostic test on the patient to diagnose the patient with the hepatitis C virus infection, and orally administering to a patient a therapeutically effective amount of a compound of Formula I:

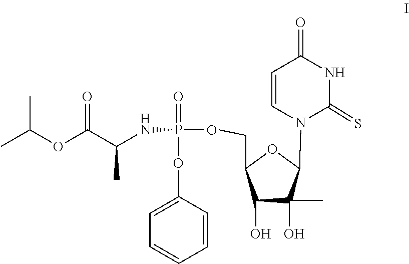

or a pharmaceutically acceptable salt thereof.

In some embodiments, the diagnostic test includes aa blood test, which may be an antibody test. The diagnostic test may evaluate the presence (or absence) of antibodies to the hepatitis C virus in the patient's blood. In some embodiments, the diagnostic test is a rapid test. In other embodiments, the diagnostic test is a standard laboratory test.

In some embodiments, diagnosing the patient with the hepatitis C virus infection includes observing a positive, or reactive, result to the diagnostic test. Such a result may indicate that the patient has, or previously had, the hepatitis C virus infection.

In some embodiments, if the patient receives a positive result on the antibody test, diagnosing the patient with the hepatitis C virus infection further includes performing a nucleic acid test on the patient, which may also be referred to as a PCR test. In some embodiments, the patient may receive a negative result on the PCR test. A negative result means that the patient had the hepatitis C virus infection previously, but the patient has been cured (by any method of treating, including the methods disclosed herein), or the hepatitis C virus infection has cleared naturally. A positive result on the PCR test means that the patient currently has the hepatitis C virus infection, without wishing to be bound by theory. If a patient receives a positive result on the antibody test and the PCR test, the patient may be treated for the hepatitis C virus infection according to any embodiment described herein.

In some embodiments, the patient may receive a negative result on the diagnostic test and/or the PCR test. In such embodiments, the patient is not treated for the hepatitis C virus infection.

There is provided a kit for treating a hepatitis C virus infection in a patient in need thereof, which includes a diagnostic test for diagnosing the patient with the hepatitis C virus infection, and a course of a pharmaceutical composition for treating the hepatitis C virus infection in the patient.

In some embodiments, the diagnostic test includes an antibody test, a PCR test, or combinations thereof. In some embodiments, the patient may take (or be administered by a clinician) the antibody test to determine if the patient has antibodies to the hepatitis C virus in the patient's blood. If the patient receives a positive result on the antibody test (indicating the presence of antibodies to the hepatitis C virus in the patient's blood), the patient may then take (or be administered by a clinician) the PCR test. If the patient receives a positive result on the PCR test (indicating that the patient currently has hepatitis C virus infection), the patient may take (or be administered by a clinician) the course of the pharmaceutical composition for treating the hepatitis C virus infection.

In some embodiments, the course of the pharmaceutical composition includes a 2 week to 24 week supply of a therapeutically effective amount of a compound of Formula I:

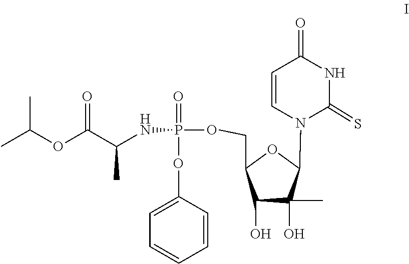

or a pharmaceutically acceptable salt thereof.

In some embodiments, the therapeutically effective amount of the compound of Formula I or a pharmaceutically acceptable salt thereof is any amount as described herein, such as about 500 mg to about 2000 mg, or any range or value contained therein. For example, the course of the pharmaceutical composition may include an amount of the compound of Formula I or a pharmaceutically acceptable salt thereof in an amount sufficient to administer the therapeutically effective amount of the compound of Formula I or a pharmaceutically acceptable salt thereof to the patient for a period of about 2 weeks to about 24 weeks. In some embodiments, the course of the pharmaceutical composition may include about 7000 mg to about 336,000 mg of the compound of Formula I or a pharmaceutically acceptable salt thereof.

In some embodiments, the therapeutically effective amount of the compound of Formula I or a pharmaceutically acceptable salt thereof is administered orally. In some embodiments, the therapeutically effective amount of the compound of Formula I or a pharmaceutically acceptable salt thereof is administered once a day. In some embodiments, the therapeutically effective amount of the compound of Formula I or a pharmaceutically acceptable salt thereof is administered twice a day.

In some embodiments, the compounds or pharmaceutically acceptable salts thereof described may be formulated as pharmaceutical compositions comprising, for example the compound of Formula I or a pharmaceutically acceptable salt thereof and a pharmaceutically acceptable excipient.

The routes for administration (delivery) of the compounds or pharmaceutically acceptable salts thereof and pharmaceutical compositions of the present methods include oral delivery (e.g., as a tablet, capsule, or as an ingestible solution). Therefore, the compounds and compositions for use in the methods of the present disclosure include those in a form especially formulated for the mode of administration. In certain embodiments, the compounds or pharmaceutically acceptable salts thereof and pharmaceutical compositions of the methods of the present disclosure are formulated in a form that is suitable for oral delivery.

The methods of the present disclosure may include compounds or pharmaceutically acceptable salts thereof and pharmaceutical compositions formulated for administration in any convenient way for use in human or veterinary medicine and the disclosure therefore includes within its scope pharmaceutical compositions comprising a compound of the disclosure adapted for use in human or veterinary medicine. Such compounds or pharmaceutically acceptable salts thereof and compositions may be presented for use in a conventional manner with the aid of one or more suitable carriers. Acceptable carriers for therapeutic use are well-known in the pharmaceutical art, and are described, for example, in Remington's Pharmaceutical Sciences, Mack Publishing Co. (A. R. Gennaro edit. 1985). The choice of pharmaceutical carrier can be selected with regard to the intended route of administration and standard pharmaceutical practice. The pharmaceutical compositions may include, in addition to the carrier, any suitable binder(s), lubricant(s), suspending agent(s), coating agent(s), and/or solubilizing agent(s).

There may be different composition/formulation requirements depending on the different delivery systems. It is to be understood that not all of the compounds need to be administered by the same route. Likewise, if the composition includes more than one active component, then those components may be administered by different routes. By way of example, the compounds or pharmaceutically acceptable salts thereof and pharmaceutical compositions of the disclosure may be formulated to be delivered using a mini pump or by a mucosal route, for example, as a nasal spray or aerosol for inhalation or ingestible solution, or parenterally in which the compounds or pharmaceutically acceptable salts thereof or composition is formulated by an injectable form, for delivery by, for example, an intravenous, intramuscular or subcutaneous route. Alternatively, the formulation may be designed to be delivered by multiple routes.

Where appropriate, the compounds or pharmaceutically acceptable salts thereof and pharmaceutical compositions can be administered by inhalation, by use of a skin patch, orally in the form of tablets containing excipients such as starch or lactose, or in capsules or ovules either alone or in admixture with excipients, or in the form of elixirs, solutions, or suspensions containing flavoring or coloring agents, or they can be injected parenterally, for example intravenously, intramuscularly, or subcutaneously. For buccal or sublingual administration, the compositions may be administered in the form of tablets or lozenges, which can be formulated in a conventional manner.

The compounds or pharmaceutically acceptable salts thereof and pharmaceutical compositions of the present disclosure can be administered in the form of tablets, capsules, troches, ovules, elixirs, solutions, or suspensions, for immediate-, delayed-, modified-, sustained-, pulsed-, or controlled-release applications. The compounds or pharmaceutically acceptable salts thereof and pharmaceutical compositions of the present disclosure may also be presented in the form of solutions, gels, syrups, or suspensions, or a dry powder for reconstitution with water or other suitable vehicle before use. Solid compositions such as tablets, capsules, lozenges, troches, pastilles, pills, boluses, powder, pastes, granules, bullets, or premix preparations may also be used. Solid and liquid compositions for oral use may be prepared according to methods well-known in the art. Such compositions may also contain one or more pharmaceutically acceptable carriers and excipients which may be in solid or liquid form.

Oral preparations may optionally include various standard pharmaceutical carriers and excipients, such as binders, fillers, buffers, lubricants, glidants, dyes, disintegrants, odorants, sweeteners, surfactants, mold release agents, antiadhesive agents, and coatings. Some excipients may have multiple roles in the compositions, e.g., act as both binders and disintegrants.

The compounds or pharmaceutically acceptable salts thereof and pharmaceutical composition or unit dosage form of the disclosure may be administered according to a dosage and administration regimen defined by routine testing in the light of the guidelines given above in order to obtain optimal activity while minim/zing toxicity or side effects for a particular subject. However, such fine tuning of the therapeutic regimen is routine in the light of the guidelines given herein.

The dosage of the compounds of the disclosure may vary according to a variety of factors such as underlying disease conditions, the individual's condition, weight, sex and age, and the mode of administration. An effective amount for treating a disorder can easily be determined by empirical methods known to those of ordinary skill in the art, for example by establishing a matrix of dosages and frequencies of administration and comparing a group of experimental units or subjects at each point in the matrix. The exact amount to be administered to a subject will vary depending on the state and severity of the disorder and the physical condition of the subject. A measurable amelioration of any symptom or parameter can be determined by a person skilled in the art or reported by the subject to the physician. It will be understood that any clinically or statistically significant attenuation or amelioration of any symptom or parameter of urinary tract disorders is within the scope of the disclosure. Clinically significant attenuation or amelioration means perceptible to the subject and/or to the physician. It will be understood that the pharmaceutical formulations of the disclosure need not necessarily contain the entire amount of the compound that is effective in treating the disorder, as such effective amounts can be reached by administration of a plurality of divided doses of such pharmaceutical formulations.

### EXAMPLES

The following examples were carried out according to embodiments of the present disclosure.

### Example 1

Göttingen pigs (“minipigs”) were given a single oral dose of the compound of Formula I at 125 mg/kg or a single intravenous bolus dose of the compound of Formula I at 25 mg/kg. Tissue samples were collected at 3, 12, and 24 hours post-dose (hpd) for the oral dose and 2 and 12 hpd for the intravenous dose, snap-frozen in liquid nitrogen, and stored at −60 to −90° C. until shipment on dry ice for analysis by LC-MS/MS. Tissue concentrations of the dosing prodrug the compound of Formula I, its M1 metabolite the compound of Formula II, the monophosphate metabolite the compound of Formula III, and the triphosphate the compound of Formula V, as well as a non-active nucleoside metabolite the compound of Formula VI were quantified using qualified bioanalytical methods.

After intravenous (IV) dosing with the compound of Formula I, the compound of Formula I was observed at 2 hpd in all tissues except the brain, with the highest concentration in the heart at 112±69.0 ng/g and the lowest concentration in the liver at 7.03±8.24 ng/g. At 12 hpd the compound of Formula I was detectable only in the heart at 2.95±5.90 ng/g but not in any other tissues. After oral dosing with the compound of Formula I, the compound of Formula I was detectable at 3 hpd in all tissues except the kidney, ranging from 270±361 ng/g in the liver to 31.5±63.0 ng/g in the brain. the compound of Formula I was not observed in tissues at the 12- or 24-h timepoints after the oral dose.

After intravenous dosing with the compound of Formula I, its M1 metabolite, the compound of Formula II, was observed at 2 hpd in all tissues, with concentrations ranging from 6920±3290 ng/g in the kidney to 4.90±9.80 ng/g in the brain. The compound of Formula II was not observable at the 12 h timepoint in the brain after intravenous dosing but was present in all other tissues, ranging from 39.3±32.1 ng/g in the kidney to 7.85±9.50 ng/g in the heart. After oral dosing with the compound of Formula I, the compound of Formula II was observed at 3 hpd in all tissues, with low levels in the brain at 27.0±23.4 ng/g, intermediate levels in the heart, lung, and spleen at 780±375 ng/g, 1630±667 ng/g, and 1390±301 ng/g, respectively, and the highest levels in the kidney and liver at 10300±2300 ng/g and 12400±5150 ng/g, respectively. The compound of Formula II was not detectable in the brain at later timepoints after oral dosing, but was observed in the heart up to 12 hpd and up to 24 hpd for all other tissues, with the highest final concentration in the liver at 45.6±25.8 ng/g.

After intravenous dosing with the compound of Formula I, its 5′-monophosphate metabolite, the compound of Formula III, was not observed in the brain and was detectable in the heart only at 2 hpd, with a concentration of 89.9±65.5 ng/g. In other organs at 2 hpd the highest concentrations were in the liver at 13100±3070 ng/g and in the kidney where it was above the limit of quantitation. At 12 hpd, the compound of Formula III was observed in all tissues except the brain and heart, with the highest concentration in the lung at 357±207 ng/g and the lowest concentration in the spleen at 58.9±14.7 ng/g. After oral dosing with the compound of Formula I, the compound of Formula III was not observed in the brain and was detectable in the heart and spleen up to 3 hours post-dose with concentrations of 34.0±39.9 ng/g and 118±10.8 ng/g, respectively. The highest concentrations at 3 hpd after oral dosing were in the kidney at 14100±5050 ng/g and in the liver, which was above the limit of quantitation. At 24 hpd, the compound of Formula III was observed only in the kidney and liver, with concentrations of 85.5±18.1 ng/g and 190±81.0 ng/g, respectively.

At 2 hpd after intravenous dosing with the compound of Formula I, the 5′-triphosphate metabolite, the compound of Formula V, had the highest level in the kidney, with concentrations of 4410±2040 ng/g, but it was not detectable in the kidney at 12 hpd. In the liver, lung, heart and spleen the compound of Formula V levels at 2 hpd were 2610±892, 3620±2770, 405±149, and 318±181 ng/g, respectively, which decreased at 12 hpd to 62.8±126, 455±263, 140±32.0, and 47.5±95.0 ng/g, respectively. The compound of Formula V was not observed in the brain at any time.

At 3 hpd after oral dosing with the compound of Formula I, the compound of Formula V levels were highest in the liver at 6670±6310 ng/g, and were low in heart, kidney and lung at 64.3±75.4 ng/g, 613±280 ng/g, and 170±29.5 ng/g, respectively, and were not detected in the brain and spleen. At 12 hpd after oral dosing only the liver had detectable EIDD-1992 at 452±541 ng/g. No tissues had detectable the compound of Formula V at 24 hpd after oral dosing.

Parent nucleoside, the compound of Formula VI, is an “inactive” metabolite of the compound of Formula I. Since the compound of Formula I has good stability in minipig plasma, it is mainly generated intracellularly in minipig organs, but it can be exported to animal plasma and then secondarily imported back to tissues. At 2-3 hours after dosing, the intracellular levels of the compound of Formula VI were relatively similar after IV 25 mg/kg or PO 125 mg/kg dosing with the kidney and liver having the highest levels at approximately 10-fold higher than in the heart, lung and spleen, and additional 10-fold lower level was observed in the brain. At 12 hours post oral dose the levels of the compound of Formula VI decreased 4-6-fold in all organs and additionally decreased 2-3 fold at 24 hpd with approximately the same relative abundance between the organs. The levels of the compound of Formula VI were decreasing faster in all organs after IV dosing resulting in 12 hpd levels approximately 10-fold lower in each organ than the corresponding levels after the PO dosing.

In conclusion, after IV or oral dosing, the prodrug the compound of Formula I apparently quickly distributes to organs and is efficiently metabolized to M1- and 5′-monophosphate metabolites. 2-3 hours after IV or PO dosing it is detectable at low quantities (˜0.1 μM) in all organs and is not detectable at 12 hours post-doses. As expected, slightly higher average concentrations were detected in the liver at 3 hpd after PO dosing.

Analysis of M1 metabolite, the compound of Formula II, and 5′-monophosphate metabolite, the compound of Formula III (MP) showed that the compound of Formula I was efficiently delivered after IV dosing to all organs other than the brain, and with more exposure to kidneys. After PO dosing, more exposure to the liver and kidneys was observed, with about 10-fold lower exposure in the lung and spleen. High levels of M1 and MP in kidney and other non-liver organs after PO dosing suggests that a significant portion of the compound of Formula I was present in post-liver blood.

The 5′-triphosphate metabolite, the compound of Formula V, accumulated in the liver (and at lower levels in the kidney) after oral dosing with the compound of Formula I, and it was still present in the liver 12 hpd at ˜1 μM (assumed tissue density of 1 g/mL) but was below the quantitation level 24 hpd in all organs, even though the MP was still measurable at ˜0.5 μM in the liver and kidney; this may suggest a short half-life of the compound of Formula V in these organs. After IV dosing with the compound of Formula I, the compound of Formula V was well accumulated in the kidney and lung and was maintained at ˜1 μM in lungs at 12 hrs post-dose.

Naïve Göttingen pigs (minipigs) between approximately three to four months old, weighing between 10.5-13.5 kilograms, were acquired from Marshall Bioresources and were acclimated to their environment for fourteen days prior to dosing. Animals were weighed the day prior to dosing to determine the dosing volume. Animals were assigned for dosing and necropsy according to the parameters set forth in TABLE 1.

TABLE 1

| | | | | | Tissue |
|---|---|---|---|---|---|
| | | Dose | Dose | Dose | Collection |
| Number of | Dose | Level | Conc. | Volume | Times |
| Animals | Route | (mg/kg) | (mg/mL) | (mL/kg) | (hpd) |
| 6/sex | Oral | 125 | 25 | 5 | 3, 12, 24 |
| 4/sex | Intravenous | 25 | 12.5 | 2 | 2, 12 |

The compound of Formula I was dissolved in a PEG 400/DI water (60%/40% v/v) vehicle for oral dosing. For IV dosing the compound of Formula I was dissolved in a PEG 400/Cremophor RH 40/Saline (40%/10%/50% v/v) vehicle and passed through a sterile filter. Göttingen pigs were dosed by a single oral dose of 125 mg/kg or by a single intravenous bolus dose of 25 mg/kg. Animals were euthanized by euthanasia solution administration under sedation, if necessary (e.g., acepromazine and or/Telazol®), followed by a Testing Facility SOP approved method to ensure death (e.g., exsanguination). Tissues were collected from two animals per sex per timepoint. Tissue samples, including the brain, heart, kidney, liver, lung, spleen, pancreas, bone marrow, duodenum, jejunum, and colon, were collected in duplicate (approximately 50±20 mg each) and placed into two separate reinforced homogenization tubes per organ (one tissue sample per tube). The exact weight (in milligrams) of each sample was recorded. Tissues samples were flash frozen in liquid nitrogen and stored at −60 to −90° C. until shipment on dry ice for analysis by LC-MS/MS.

Göttingen pig tissues from the brain, heart, kidney, liver, lung, and spleen were extracted with 70% acetonitrile in water that included EIDD-2478, EIDD-2479, EIDD-2510, ¹³C₁₀¹⁵N₅ ATP, and ¹³C₉¹⁵N₂ UTP as internal standards by homogenization in a Lysera bead mill outfitted with a cryo cooling unit (Biotage, Uppsala, Sweden). To remove large solids, the homogenate was centrifuged for 5 minutes at 10,000 rpm in an Eppendorf 5425R centrifuge (Eppendorf, Hamburg, Germany). The supernatant was then transferred to a micro-centrifuge tube and centrifuged again in an Eppendorf 5425R centrifuge for 5 minutes at 15,000 rpm to remove any remaining solids. The remaining supernatant was transferred to an HPLC vial for analysis according to a qualified method described in Bioanalytical Method Report BAM-119v3, “Analysis of EIDD-2023 and its Metabolites in Animal Tissues by LCMS-MS.” HPLC separation was performed on an Agilent 1260 system (Agilent Technologies, Santa Clara, CA, USA) equipped with an autosampler, column oven, UV lamp, and binary pump. A SeQuant ZIC-PHILIC PEEK-coated (100×4.6 mm, 5 μm particle size) column (Merck Millipore, Burlington, MA, USA) was used for the separation of the compound of Formula I, the compound of Formula II, the compound of Formula III, the compound of Formula V, the compound of Formula VI, EIDD-2478, EIDD-2479, EIDD-2510, ¹³C₁₀¹⁵N₅ ATP, and ¹³C₉¹⁵N₂ UTP. Mobile phase A consisted of 50 mM ammonium bicarbonate buffer in HPLC grade water pH 9.8 and mobile phase B consisted of pure acetonitrile. A 7.3-minute HPLC method was performed to separate the analytes beginning with a 0.8-minute isocratic hold at 30% A, followed by 5-minute gradient to 55% A, and a return to starting conditions for 0.6 minutes. Mass spectrometry analysis was performed on a QTrap 7500 Mass Spectrometer (Sciex, Framingham, MA, USA) using negative mode electrospray ionization (ESI) in multiple reaction monitoring (MRM) mode. Data analysis was performed using SCIEX OS Software (Sciex, Framingham, MA, USA).

Göttingen pigs were given a single oral dose of the compound of Formula I at 125 mg/kg or a single intravenous bolus dose of the compound of Formula I at 25 mg/kg. Tissue samples were collected at 3, 12, and 24 hours post-dose for the oral dose and 2 and 12 hour post-dose for the intravenous dose. The tissue concentration profiles of the compound of Formula I in Göttingen pig tissues are shown in TABLE 2.

TABLE 2

| | Dose | | | | |
|---|---|---|---|---|---|
| Tissue | (mg/kg) | 2 hpd | 3 hpd | 12 hpd | 24 hpd |
| Brain | PO-125 | nc | 31.5 ± 63.0 | BQL | BQL |
| Heart | PO-125 | nc | 117 ± 105 | BQL | BQL |
| Kidney | PO-125 | nc | BQL | BQL | BQL |
| Liver | PO-125 | nc | 270 ± 361 | BQL | BQL |
| Lung | PO-125 | nc | 38.6 ± 46.5 | BQL | BQL |
| Spleen | PO-125 | nc | 88.4 ± 60.3 | BQL | BQL |
| Brain | IV-25 | BQL | nc | BQL | nc |
| Heart | IV-25 | 112 ± 69.0 | nc | 2.95 ± 5.90 | nc |
| Kidney | IV-25 | 85.0 ± 93.1 | nc | BQL | nc |
| Liver | IV-25 | 7.03 ± 8.24 | nc | BQL | nc |
| Lung | IV-25 | 51.8 ± 29.1 | nc | BQL | nc |
| Spleen | IV-25 | 63.9 ± 29.8 | nc | BQL | nc |

The tissue concentration profiles of the compound of Formula II in Göttingen pig tissues are shown in TABLE 3.

TABLE 3

| | Dose | | | | |
|---|---|---|---|---|---|
| Tissue | (mg/kg) | 2 hpd | 3 hpd | 12 hpd | 24 hpd |
| Brain | PO-125 | nc | 27.0 ± 23.4 | BQL | BQL |
| Heart | PO-125 | nc | 780 ± 375 | 3.33 ± 6.65 | BQL |
| Kidney | PO-125 | nc | 10300 ± 2300 | 180 ± 172 | 30.0 ± 12.8 |
| Liver | PO-125 | nc | 12400 ± 5150 | 202 ± 309 | 45.6 ± 25.8 |
| Lung | PO-125 | nc | 1630 ± 667 | 16.5 ± 12.8 | 2.25 ± 4.51 |
| Spleen | PO-125 | nc | 1390 ± 301 | 11.9 ± 9.24 | 2.27 ± 4.53 |
| Brain | IV-25 | 4.90 ± 9.80 | nc | BQL | nc |
| Heart | IV-25 | 292 ± 139 | nc | 7.85 ± 9.50 | nc |
| Kidney | IV-25 | 6920 ± 3290 | nc | 39.3 ± 32.1 | nc |
| Liver | IV-25 | 694 ± 385 | nc | 17.9 ± 16.4 | nc |
| Lung | IV-25 | 786 ± 134 | nc | 31.8 ± 5.52 | nc |
| Spleen | IV-25 | 530 ± 284 | nc | 24.8 ± 7.34 | nc |

The tissue concentration profiles of the compound of Formula III in Göttingen pig tissues are shown in TABLE 4.

TABLE 4

| | Dose | | | | |
|---|---|---|---|---|---|
| Tissue | (mg/kg) | 2 hpd | 3 hpd | 12 hpd | 24 hpd |
| Brain | PO-125 | nc | BQL | BQL | BQL |
| Heart | PO-125 | nc | 34.0 ± 39.9 | BQL | BQL |
| Kidney | PO-125 | nc | 14100 ± 5050 | 824 ± 432 | 85.5 ± 18.1 |
| Liver | PO-125 | nc | AQL | 582 ± 328 | 190 ± 81.0 |
| Lung | PO-125 | nc | 303 ± 94.4 | 55.2 ± 64.6 | BQL |
| Spleen | PO-125 | nc | 118 ± 10.8 | BQL | BQL |
| Brain | IV-25 | BQL | nc | BQL | nc |
| Heart | IV-25 | 89.9 ± 65.5 | nc | BQL | nc |
| Kidney | IV-25 | AQL | nc | 276 ± 78.1 | nc |
| Liver | IV-25 | 13100 ± 3070 | nc | 62.7 ± 77.7 | nc |
| Lung | IV-25 | 2040 ± 883 | nc | 357 ± 207 | nc |
| Spleen | IV-25 | 240 ± 28.3 | nc | 58.9 ± 14.7 | nc |

The tissue concentration profiles of the compound of Formula V in Göttingen pig tissues are shown in TABLE 5.

TABLE 5

| | Dose | | | | |
|---|---|---|---|---|---|
| Tissue | (mg/kg) | 2 hpd | 3 hpd | 12 hpd | 24 hpd |
| Brain | PO-125 | nc | BQL | BQL | BQL |
| Heart | PO-125 | nc | 64.3 ± 75.4 | BQL | BQL |
| Kidney | PO-125 | nc | 613 ± 280 | BQL | BQL |
| Liver | PO-125 | nc | 6670 ± 6310 | 452 ± 541 | BQL |
| Lung | PO-125 | nc | 170 ± 29.5 | BQL | BQL |
| Spleen | PO-125 | nc | BQL | BQL | BQL |
| Brain | IV-25 | BQL | nc | BQL | nc |
| Heart | IV-25 | 405 ± 149 | nc | 140 ± 32.0 | nc |
| Kidney | IV-25 | 4410 ± 2040 | nc | BQL | nc |
| Liver | IV-25 | 2610 ± 892 | nc | 62.8 ± 126 | nc |
| Lung | IV-25 | 3620 ± 2770 | nc | 455 ± 263 | nc |
| Spleen | IV-25 | 318 ± 181 | nc | 47.5 ± 95.0 | nc |

The tissue concentration profiles of the compound of Formula VI in Göttingen pig tissues are shown in TABLE 6.

TABLE 6

| | Dose | | | | |
|---|---|---|---|---|---|
| Tissue | (mg/kg) | 2 hpd | 3 hpd | 12 hpd | 24 hpd |
| Brain | PO-125 | nc | 192 ± 77.9 | 111 ± 38.6 | 37.9 ± 9.15 |
| Heart | PO-125 | nc | 3370 ± 645 | 533 ± 213 | 240 ± 27.7 |
| Kidney | PO-125 | nc | 29400 ± 2170 | 7420 ± 1440 | 2740 ± 503 |
| Liver | PO-125 | nc | 18200 ± 6500 | 2600 ± 1530 | 1120 ± 505 |
| Lung | PO-125 | nc | 3580 ± 1060 | 536 ± 101 | 242 ± 85.7 |
| Spleen | PO-125 | nc | 3010 ± 433 | 539 ± 195 | 292 ± 139 |
| Brain | IV-25 | 140 ± 56.7 | nc | 6.40 ± 12.8 | nc |
| Heart | IV-25 | 1990 ± 195 | nc | 57.3 ± 23.3 | nc |
| Kidney | IV-25 | 25400 ± 5560 | nc | 701 ± 48.7 | nc |
| Liver | IV-25 | 7960 ± 1530 | nc | 201 ± 126 | nc |
| Lung | IV-25 | 2330 ± 130 | nc | 93.9 ± 18.8 | nc |
| Spleen | IV-25 | 1620 ± 181 | nc | 49.2 ± 9.70 | nc |

After IV or oral dosing, the prodrug the compound of Formula I apparently quickly distributes to organs and is efficiently metabolized to M1- and 5′-monophosphate metabolites. 2-3 hours after IV or PO dosing it is detectable at low quantities (˜0.1 μM) in all organs and is not detectable at 12 hours post-doses. As expected, slightly higher average concentrations were detected in the liver at 3 hpd after PO dosing.

Analysis of M1 metabolite, the compound of Formula II, and 5′-monophosphate metabolite, the compound of Formula III (MP) showed that the compound of Formula I was efficiently delivered after IV dosing to all organs other than the brain, and with more exposure to kidneys. After PO dosing, more exposure to the liver and kidneys was observed, with about 10-fold lower exposure in the lung and spleen. High levels of M1 and MP in kidney and other non-liver organs after PO dosing suggests that a significant portion of the compound of Formula I was present in post-liver blood.

The 5′-triphosphate metabolite, the compound of Formula V, accumulated in the liver (and at lower levels in the kidney) after oral dosing with the compound of Formula I, and it was still present in the liver 12 hpd at ˜1 μM (assumed tissue density of 1 g/mL) but was below the quantitation level 24 hpd in all organs, even though the MP was still measurable at ˜0.5 μM in the liver and kidney; this may suggest a short half-life of the compound of Formula V in these organs. After IV dosing with the compound of Formula I, the the compound of Formula V was well accumulated in the kidney and lung and was maintained at ˜1 μM in lungs at 12 hrs post-dose.

TABLE 7 shows the tissue concentrations of the compound of Formula I and its metabolites in Göttingen pig liver after a single oral dose of the compound of Formula I at 125 mg/kg.

TABLE 7

| Analyte | 3 hpd | 12 hpd | 24 hpd |
|---|---|---|---|
| Formula I | 0.496 ± 0.663 | BQL | BQL |
| Formula II | 29.2 ± 12.2 | 0.475 ± 0.727 | 0.107 ± 0.0606 |
| Formula III | AQL | 1.64 ± 0.924 | 0.537 ± 0.229 |
| Formula V | 12.9 ± 12.3 | 0.880 ± 1.05 | BQL |
| Formula VI | 66.3 ± 23.7 | 9.46 ± 5.56 | 4.08 ± 1.84 |

This disclosure is not limited to the particular systems, devices and methods described, as these may vary. The terminology used in the description is for the purpose of describing the particular versions or embodiments only and is not intended to limit the scope.

In the above detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be used, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (for example, bodies of the appended claims) are generally intended as “open” terms (for example, the term “including” should be interpreted as “including but not limited to,” the term “having” should be interpreted as “having at least,” the term “includes” should be interpreted as “includes but is not limited to,” et cetera). While various compositions, methods, and devices are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups. It will be further understood by those within the art that if a specific number of an introduced claim recitation is intended, such an intent will be explicitly recited in the claim, and in the absence of such recitation no such intent is present.

For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (for example, “a” and/or “an” should be interpreted to mean “at least one” or “one or more”); the same holds true for the use of definite articles used to introduce claim recitations.

In addition, even if a specific number of an introduced claim recitation is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, et cetera” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, et cetera). In those instances where a convention analogous to “at least one of A, B, or C, et cetera” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, et cetera). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, et cetera. As a non-limiting example, each range discussed herein can be readily broken down into a lower third, middle third and upper third, et cetera. As will also be understood by one skilled in the art all language such as “up to,” “at least,” and the like include the number recited and refer to ranges that can be subsequently broken down into subranges as discussed above. Finally, as will be understood by one skilled in the art, a range includes each individual member. Thus, for example, a group having 1-3 compounds refers to groups having 1, 2, or 3 compounds. Similarly, a group having 1-5 cells refers to groups having 1, 2, 3, 4, or 5 compounds, and so forth.

Various of the above-disclosed and other features and functions, or alternatives thereof, may be combined into many other different systems or applications. Various presently unforeseen or unanticipated alternatives, modifications, variations, or improvements therein may be subsequently made by those skilled in the art, each of which is also intended to be encompassed by the disclosed embodiments.

### EMBODIMENTS

[symbol]

What is claimed is:

1. A method of treating a hepatitis C virus infection in a patient in need thereof comprising orally administering to the patient in need thereof, a therapeutically effective amount of a compound of Formula I: 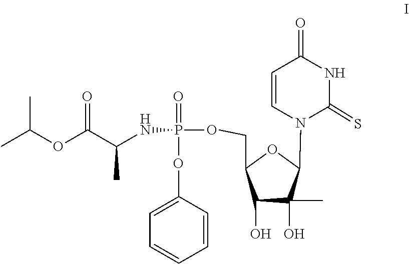
or a pharmaceutically acceptable salt thereof, thereby treating the hepatitis C virus infection in the patient.

2. The method of claim 1, wherein the therapeutically effective amount of the compound of Formula I or a pharmaceutically acceptable salt thereof is between about 5 mg/kg and about 400 mg/kg.

3. The method of claim 1, wherein the therapeutically effective amount of the compound of Formula I or a pharmaceutically acceptable salt thereof is about 25 mg/kg.

4. The method of claim 1, wherein the therapeutically effective amount of the compound of Formula I or a pharmaceutically acceptable salt thereof is between about 500 mg and about 2000 mg.

5. The method of claim 1, wherein the therapeutically effective amount of the compound of Formula I or a pharmaceutically acceptable salt thereof is administered once a day.

6. The method of claim 1, wherein the therapeutically effective amount of the compound of Formula I or a pharmaceutically acceptable salt thereof is administered twice a day.

7. The method of claim 1, wherein the therapeutically effective amount of the compound of Formula I or a pharmaceutically acceptable salt thereof is administered over a period of about 2 weeks to about 24 weeks.

8. The method of claim 1, wherein the hepatitis C virus infection is selected from the group consisting of Genotype 1, Genotype 2, Genotype 3, Genotype 4, Genotype 5, or Genotype 6, or any combination thereof.

9. The method of claim 1, wherein the hepatitis C virus infection is a strain selected from the group consisting of 1a, 1b, 1b/2b NS5B, 1b/4a NS5B, 1b/NS5B S282T, 1b/NS5B S96T, 1b/3a NS5B, or any combination thereof.

10. The method of claim 1, wherein the patient has compensated cirrhosis.

11. The method of claim 1, wherein the patient has decompensated cirrhosis.

12. The method of claim 1, wherein the patient does not have cirrhosis.

13. The method of claim 1, wherein administering the therapeutically effective amount of the compound of Formula I or a pharmaceutically acceptable salt thereof results in clearance of the hepatitis C virus infection in the patient.

14. The method of claim 1, wherein administering the therapeutically effective amount of the compound of Formula I or a pharmaceutically acceptable salt thereof results in a sustained virologic response after about 12 weeks to about 24 weeks of treatment.

15. The method of claim 1, further comprising administering a second active agent for the treatment of the hepatitis C virus infection.

16. The method of claim 15, wherein the second active agent for the treatment of the hepatitis C virus infection comprises sofosbuvir, ledipasvir, velpatasvir, elbasvir, grazoprevir, glecaprevir, pibrentasvir, voxelaprevir, ribavirin, boceprevir, daclatasvir, ombitasvir, paritaprevir, ritonavir, dasabuvir, simeprevir, telaprevir, or combinations thereof.

17. The method of claim 15, further comprising administering a third active agent for the treatment of the hepatitis C virus infection.

18. The method of claim 17, wherein the third active agent for the treatment of the hepatitis C virus infection comprises sofosbuvir, ledipasvir, velpatasvir, elbasvir, grazoprevir, glecaprevir, pibrentasvir, voxelaprevir, ribavirin, boceprevir, daclatasvir, ombitasvir, paritaprevir, ritonavir, dasabuvir, simeprevir, telaprevir, or combinations thereof.

19. A method of treating a hepatitis C virus infection in a patient in need thereof comprising orally administering to the patient a therapeutically effective amount of a compound of Formula I: 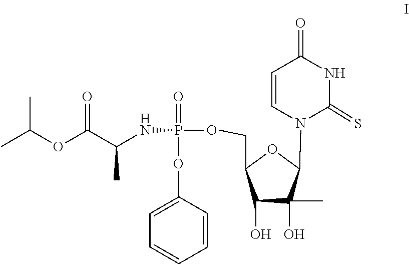
or a pharmaceutically acceptable salt thereof, wherein administering a therapeutically effective amount of the compound of Formula I or a pharmaceutically acceptable salt thereof results in clearance of the hepatitis C virus infection.

20. The method of claim 19, wherein clearance of the hepatitis C virus infection comprises a sustained virologic response after about 12 weeks to about 24 weeks of treatment.

21. The method of claim 19, wherein the therapeutically effective amount of the compound of Formula I or a pharmaceutically acceptable salt thereof is between about 5 mg/kg and about 400 mg/kg.

22. The method of claim 19, wherein the therapeutically effective amount of the compound of Formula I or a pharmaceutically acceptable salt thereof is about 25 mg/kg.

23. The method of claim 19, wherein the therapeutically effective amount of the compound of Formula I or a pharmaceutically acceptable salt thereof is administered once a day.

24. The method of claim 19, wherein the therapeutically effective amount of the compound of Formula I or a pharmaceutically acceptable salt thereof is administered twice a day.

25. The method of claim 19, wherein the hepatitis C virus infection is selected from the group consisting of Genotype 1, Genotype 2, Genotype 3, Genotype 4, Genotype 5, or Genotype 6, or any combination thereof.

26. The method of claim 19, wherein the hepatitis C virus infection is a strain selected from the group consisting of 1a, 1b, 1b/2b NS5B, 1b/4a NS5B, 1b/NS5B S282T, 1b/NS5B S96T, 1b/3a NS5B, or any combination thereof.

27. The method of claim 19, wherein the patient has compensated cirrhosis.

28. The method of claim 19, wherein the patient has decompensated cirrhosis.

29. The method of claim 19, wherein the patient does not have cirrhosis.

30. The method of claim 19, further comprising administering a second active agent for the treatment of the hepatitis C virus infection.

31. The method of claim 30, wherein the second active agent for the treatment of the hepatitis C virus infection comprises sofosbuvir, ledipasvir, velpatasvir, elbasvir, grazoprevir, glecaprevir, pibrentasvir, voxelaprevir, ribavirin, boceprevir, daclatasvir, ombitasvir, paritaprevir, ritonavir, dasabuvir, simeprevir, telaprevir, or combinations thereof.

32. The method of claim 30, further comprising administering a third active agent for the treatment of the hepatitis C virus infection.

33. The method of claim 32, wherein the third active agent for the treatment of the hepatitis C virus infection comprises sofosbuvir, ledipasvir, velpatasvir, elbasvir, grazoprevir, glecaprevir, pibrentasvir, voxelaprevir, ribavirin, boceprevir, daclatasvir, ombitasvir, paritaprevir, ritonavir, dasabuvir, simeprevir, telaprevir, or combinations thereof.

34. A method of treating a hepatitis C virus infection in a patient in need thereof comprising orally administering to the patient a prodrug of Formula I: 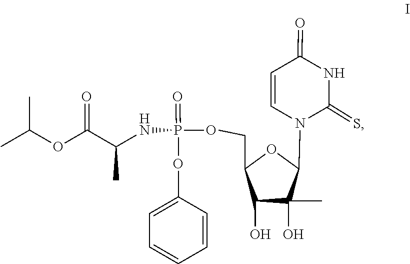
or a pharmaceutically acceptable salt thereof; wherein the prodrug is administered in an amount sufficient to result in a therapeutically effective amount of one or more of a compound of Formula II-VI in the liver of the patient: 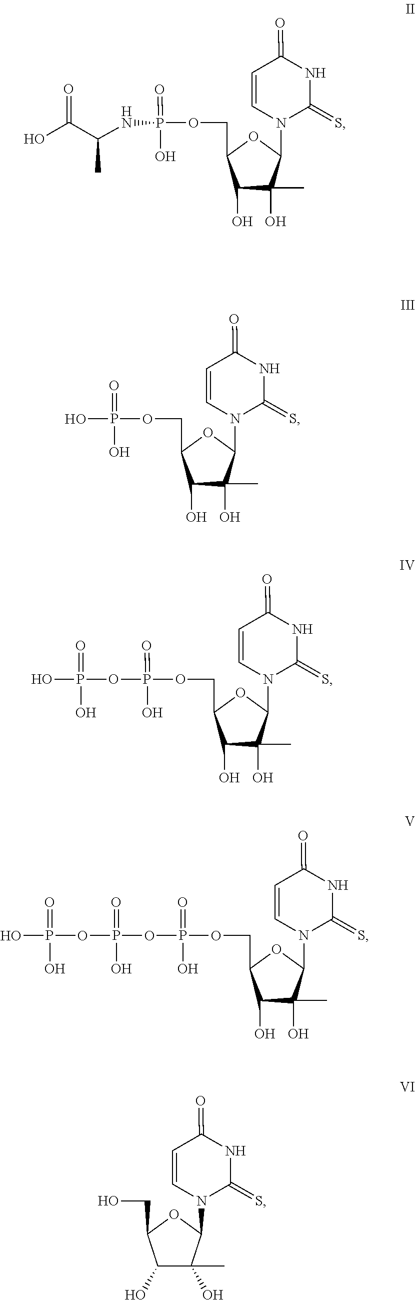
thereby treating the hepatitis C virus infection in the patient.

35. The method of claim 34, wherein the amount of the prodrug of Formula I or a pharmaceutically acceptable salt thereof is between about 5 mg/kg and about 400 mg/kg.

36. The method of claim 34, wherein the amount of the prodrug of Formula I or a pharmaceutically acceptable salt thereof is about 25 mg/kg.

37. The method of claim 34, wherein the prodrug of Formula I or a pharmaceutically acceptable salt thereof is administered once a day.

38. The method of claim 34, wherein the prodrug of Formula I or a pharmaceutically acceptable salt thereof is administered twice a day.

39. The method of claim 34, wherein the prodrug of Formula I or a pharmaceutically acceptable salt thereof is administered over a period of about 2 weeks to about 24 weeks.

40. The method of claim 34, wherein the hepatitis C virus infection is selected from the group consisting of Genotype 1, Genotype 2, Genotype 3, Genotype 4, Genotype 5, Genotype 6, or any combination thereof.

41. The method of claim 34, wherein the hepatitis C virus infection is a strain selected from the group consisting of 1a, 1b, 1b/2b NS5B, 1b/4a NS5B, 1b/NS5B S282T, 1b/NS5B S96T, 1b/3a NS5B, or any combination thereof.

42. The method of claim 34, wherein the patient has compensated cirrhosis.

43. The method of claim 34, wherein the patient has decompensated cirrhosis.

44. The method of claim 34, wherein the patient does not have cirrhosis.

45. The method of claim 34, wherein administering the prodrug of Formula I or a pharmaceutically acceptable salt thereof results in clearance of the hepatitis C virus infection.

46. The method of claim 34, wherein administering the prodrug of Formula I or a pharmaceutically acceptable salt thereof results in a sustained virologic response after about 12 weeks to about 24 weeks of treatment.

47. The method of claim 34, further comprising administering a second active agent for the treatment of the hepatitis C virus infection.

48. The method of claim 47, wherein the second active agent for the treatment of the hepatitis C virus infection comprises sofosbuvir, ledipasvir, velpatasvir, elbasvir, grazoprevir, glecaprevir, pibrentasvir, voxelaprevir, ribavirin, boceprevir, daclatasvir, ombitasvir, paritaprevir, ritonavir, dasabuvir, simeprevir, telaprevir, or combinations thereof.

49. The method of claim 47, further comprising administering a third active agent for the treatment of the hepatitis C virus infection.

50. The method of claim 49, wherein the third active agent for the treatment of the hepatitis C virus infection comprises sofosbuvir, ledipasvir, velpatasvir, elbasvir, grazoprevir, glecaprevir, pibrentasvir, voxelaprevir, ribavirin, boceprevir, daclatasvir, ombitasvir, paritaprevir, ritonavir, dasabuvir, simeprevir, telaprevir, or combinations thereof.
